(19) United States
(12) Patent Application Publication
(10) Pub. No.: US 2011/0269629 A1
(43) Pub. Date: Nov. 3, 2011

(54) SUPERCONDUCTING MATERIALS

(75) Inventors: Feliciano Giustino, Oxford (GB); Andrea C. Ferrari, Cambridge (GB); Gianluca Savini, Cambridge (GB)

(73) Assignee: ISIS INNOVATION LIMITED, Oxford (GB)

(21) Appl. No.: 13/019,779

(22) Filed: Feb. 2, 2011

(30) Foreign Application Priority Data
Mar. 18, 2010 (GB) 1004554.0

(51) Int. Cl. H01L 39/12; C07C 25/00; C01B 31/00; C07C 13/615; H01L 29/775; C07F 5/02; C01B 31/36; B82Y 30/00

(52) U.S. Cl. 505/100; 568/1; 568/6; 570/129; 423/291; 423/446; 585/21; 257/29; 423/446; 977/742; 977/734; 977/762; 257/E29.245

(57) ABSTRACT

Partially or fully saturated doped graphene materials are found to be superconducting. The saturation is with hydrogen or halogen. Doping is performed by substitution of carbon atoms or by applying an electric field. Diamond nano-rods are also found to be superconducting. These materials can be used in electronic devices having a gate.

### CLAIM OF PRIORITY

This application claims the benefit of priority under 35 U.S.C. §119(a) to United Kingdom patent application no. 1004554.0, filed Mar. 18, 2010, which is incorporated herein by reference in its entirety.

### TECHNICAL FIELD

The present technology relates to superconducting materials.

### BACKGROUND

The structure of graphene is shown in FIG. 5. The material is formed of carbon atoms covalently bonded in tessellated hexagons, each atom being a vertex of 3 hexagons. The carbon atoms are sp² bonded with the electrons of the remaining p orbital being delocalised over the sheet in so called pi-bonding. Materials comprised of graphene include single sheet graphene, which for example may be in suspended membrane form or as a layer on a substrate, graphite, which is formed of many layers of graphene stacked up, and tubes, including nano-tubes, in which the sheet of graphene is wrapped around and joins seamlessly to itself. Sheets of graphene may be extensive or may be in ribbons a few nm wide.

Many methods for forming these materials are known in the prior art.

Graphene is not known to be a superconductor. Indeed it is an insulator. It is known to dope graphene to provide it with ordinary conductivity by introducing carriers, in a similar manner to semiconductor materials. Boron substituted for carbon atoms is used to provide holes and nitrogen for electrons.

Bulk diamond is known to be superconducting but only at the very low temperature of 4 K.

The discussion of the background herein is included to explain the context of the technology. This is not to be taken as an admission that any of the material referred to was published, known, or part of the common general knowledge as at the priority date of any of the claims found appended hereto.

Throughout the description and claims of the specification the word “comprise” and variations thereof, such as “comprising” and “comprises”, is not intended to exclude other additives, components, integers or steps.

### SUMMARY

The technology described herein provides materials which are superconducting.

According to the instant disclosure, there is provided a doped, at least partially saturated graphene wherein the saturation is by hydrogen, and/or halogen. This may be fully saturated by the hydrogen and/or halogen. Alternatively the proportion of carbon atoms having a hydrogen and/or a halogen may be above 75%, or 90% or 95% or 99%. The disclosure also provides for saturation by a combination of hydrogen and halogen atom(s) of various characters, in proportions as described elsewhere herein.

The substance may be in form of a sheet, or a nano-tube, or in multiple layers, or a ribbon.

The substance may be on a substrate.

The saturation may be by hydrogen.

The saturation may be by fluorine.

The hydrogen or halogen may be on one side of the carbon sheet of the graphene. The hydrogen or halogen may be on different sides of the carbon sheet of the graphene

According to the instant disclosure there is also provided a doped diamond nano-rod.

The doping may be chemical. The doping may be where carbon atoms are substituted by boron atoms. The doping may make the material p-type. The doping may make the material n-type. The doping may be by an electric field.

According to the instant disclosure there is further provided a device comprising:
a layer of at least partially saturated graphene wherein the saturation is by hydrogen and/or halogen, and
a gate layer thereon.

According to the instant disclosure there is also provided a device comprising
a diamond nano-rod, and
a gate layer thereon.

The devices may have an insulating between the gate and the nano-rod or the graphene layer.

According to the instant disclosure there is further provided a substance having a carbon structure in which carbon atoms are bonded together in by sp³ bonds, and which the carbon structure is low dimensional, exhibiting superconductivity. Low dimensional means where the conduction is confine to one or two dimensions. The carbon atoms may be arranged in sheets and the sheets may be in the form of nano-tubes. The carbon atoms may be arranged in the diamond structure.

The instant disclosure further provides superconducting power conductors comprised of the said at least partially doped graphene or diamond nano-rods.

The instant disclosure further provides superconducting signal conductors comprised of the said at least partially doped graphene or diamond nano-rods.

The instant disclosure further provides superconducting main conductors comprised of the said at least partially doped graphene or diamond nano-rods.

The instant disclosure also provides a superconducting magnet comprised of conductors comprised of the said at least partially doped graphene or diamond nano-rods.

The said at least partially doped graphene or diamond nano-rods may be comprised in a cryogenic apparatus arranged to cool the at least partially doped graphene or diamond nano-rods below it superconducting critical temperature. The material may be then used to conduct electricity, for example to convey power or a signal.

The said at least partially doped graphene or diamond nano-rods may be comprised in a cryogenic apparatus arranged to cool the at least partially doped graphene or diamond nano-rods below it superconducting critical temperature. The material may be then used to conduct electricity, for example to convey power or a signal.

All the said substances, devices, magnets, apparatus and conductors, or a part thereof, may be at a temperature below the superconducting critical temperature of the substance.

All the said substances, devices, magnets, apparatus and conductors may be characterised by having a superconducting critical temperature below room temperature.

All the said substances, devices, magnets, apparatus and conductors may be characterised by having a superconducting critical temperature above the temperature of liquid nitrogen.

All the said substances, devices, magnets, apparatus and conductors may be characterised by having a superconducting critical temperature below room temperature.

The instant disclosure also provides a method of conveying power comprising the steps of providing a conductor of a said substances, cooling the substance to below its critical temperature and passing electricity along the conductor.

The instant disclosure also provides a method of conveying a signal comprising the steps of providing a conductor of a said substances, cooling the substance to below its critical temperature and passing an electric signal along the conductor.

The instant disclosure includes a class of materials have a sheet of carbon atoms having the same basic graph of bonding in hexagons as in graphene. The carbon atoms have, however, or a proportion of them have, a respective extra bonded atom, (for example hydrogen, fluorine etc) indeed preferably a high proportion have an additional bonded atom. This means that at least those carbon atoms are sp³ bonded. Therefore generally the sheet of carbon atoms is not perfectly flat but carbon atoms are at different heights with respect to a smooth geometrical surface passing through the carbon structure (which surface may be a plane in the case of a flat sheet of graphene or a circular cylinder in the case of a carbon nano-tube). In the case of all the carbon atoms having an extra bonded atom the conformation of the sheet will generally be the chair conformation.

The extra bonded atoms envisaged are hydrogen, and halogens. Of the halogens fluorine is preferred. It also possible to have mixed atoms from those, for example some of the extra bonded atoms may be hydrogen with others being fluorine, or some may be one halogen and others another halogen, or some may be hydrogen with others being one halogen and still others being another halogen. Of the halogens chlorine, bromine and iodine may also be possible but it is not yet determined whether they will bond to the carbon sheet in the same way as fluorine. Also full population with larger halogens may not be possible due to their size.

The modified graphene structure of the disclosure may be in various forms. These include flat sheets, for example, those on a substrate, suspended membranes, or otherwise; ribbons; carbon nano-tubes of any diameter or length, single or multiply walled; and also in structures of multiple layers of graphane (for example prepared from graphite, which of course has a structure that is multiple layers of graphene).

Following the inventors' investigations, it is found that these materials are superconducting. These investigations which involve modelling to predict superconducting critical temperatures are discussed towards the end of this description of the technology. Fully hydrogenated and fully fluorinated graphene sheets have been modelled. p-type versions of these have been predicted to have much higher critical temperatures than n-type ones. A high critical temperature is important because it reduces the costs of cooling. Critical temperatures in excess of 77 K are particularly advantageous since above that temperature relatively inexpensive liquid nitrogen can be used as a coolant.

The sheets modelled have the added hydrogen or fluorine on alternating sides of the sheet of carbon atoms. i.e. for each carbon atom and its nearest neighbour the added atoms are on opposite sides. Note however sheets with the added atoms all on the same side of the sheet are possible, for example, such a structure can be formed if a graphene sheet on a substrate is hydrogenated, fluorinated. Similarly a one sided structure might form in the hydrogenation, halogenation etc of carbon nano-tubes. All these are expected also to exhibit superconductivity, owing to the sp³ bonding of the carbon sheets. Compared to hydrogenated graphene fluorinated graphane has a much lower superconducting critical temperature. However since fluorination of graphite, which itself is cheap, is a developed technique it may prove to be a cheap way of providing a cheap low temperature superconductor. Also if any of the materials prove difficult to fully hydrogenate a step of fluorination to complete the conversion to sp³ bonds may then provide a material with a superconducting critical temperature between that of the fully hydrogenated and the fully fluorinated versions.

The instant disclosure also includes diamond nano-rods; here the carbon atoms are also sp³bonded so have the same general The inventors' investigations predict a superconducting critical temperature for diamond nano-rods having a diameter of 1 nm to be 150 K. The literature already reports production of diamond nano-rods of 2 nm in diameter and for this size the inventors also expect a high critical temperature.

Diamond is already sp³ bonded so the question of the carbon structure having additional bonded atoms does not arise like it does for the structures discussed above, which in effect are being compared to graphene. A diamond nano-rod is however a low-dimensional structure, which of course bulk diamond is not and this boosts its superconducting critical temperature compared to bulk diamond.

To exhibit superconductivity the materials of the instant disclosure need to be doped to provide carriers. This may be done using chemical doping, for example by substitution of carbon atoms with boron for p-type or nitrogen for n-type. Alternatively it may be done by applying an electric field, for example with an electrode, which would be termed a gate in many geometries.

Clearly also, of course, to exhibit superconductivity the materials have to be cooled to below their superconducting critical temperature, as that term is understood in the art.

The materials provided by the technology herein may be used as conductors, for example in microcircuits or even possibly as bulk conductors. They may also be used as the active regions in electronic devices such as transistors.

Generally the materials of the instant disclosure may be made using techniques analogous to known techniques.

Starting materials for the preparation of materials of the instant disclosure include graphene, graphite and carbon nano-tubes.

Graphene may be prepared by various methods, for example, by mechanical exfoliation on Si/SiO₂ of highly oriented pyrlitic graphite [for example, K. S. Novoselov et al., Proc. Natl. Acad. Sci. U.S.A. 102, 10451 (2005)], or alternatively by graphitization of clean surfaces of commercial SiC wafers [for example, C. Berger et al, Science 312, 1191 (2006), which is incorporated herein by reference]. Wafer-size production processes on SiC(0001) surfaces have been developed [for example, K. V. Emtsev et al, Nature Materials 8, 203 (2009), which is incorporated herein by reference]. Wafer-size production processes on Cu foils has also been demonstrated, using CVD growth of carbon using a mixture of methane and hydrogen [for example, X. Li et al, Science 324, 1312 (2009), which is incorporated herein by reference].

Carbon nanotubes may be produced by a number of techniques, from arc discharge of graphite electrodes to laser ablation of graphite.

Diamond nano-rods may be fabricated by various methods. One method is CVD using methane, [for example N. Shang et al, ACS Nano 3, 1032 (2009), which is incorporated herein by reference] Fabrication of diamond nano-rods with diameter as small as 4 nm is, for example, described in [L. T. Sun et al, Adv. Mater. 26, 1849 (2004), which is incorporated herein by reference], the fabrication of diamond nano-rods with diameter of 2 nm is described, for example, in [N. Shang et al, ACS Nano 3, 1032 (2009), which is incorporated herein by reference].

In the preferred method if the graphene, carbon nano-tube, graphite material etc. is to be chemically doped this is carried out before it is hydrogenated, fluorinated etc.

Graphane and carbon nanotubes may be doped with boron by high temperature reaction with boron oxide (B₂O₃) [for example, see W. Han et al, Chem. Phys. Lett. 299, 368 (1999), which is incorporated herein by reference]

For diamond nano-rods boron doping can be achieved during the CVD growth of the nano-rods by mixing a boron providing species into the vapour [see for example, Y. Takano et al, Diam. Rel. Mater. 16, 911 (2007), which is incorporated herein by reference]

Boron doped graphite can be fabricated by reacting graphite with a solution of boric acid H₃BO₃ [see for example, T. Hagio et al, Carbon 27, 259 (1989), which is incorporated herein by reference].

In order to achieve n-doping graphene, carbon nanotubes or graphite can be n-doped one method is by introducing nitrogen to those materials by electrical joule heating in ammonia (NH₃) gas. This procedure has been demonstrated, for example, in [X. Wang et al., Science 8, 768 (2009), which is incorporated herein by reference].

The hydrogenation or fluorination etc of the graphane, carbon nano-tubes, graphite etc. may be achieved by the methods described below.

Graphane may be prepared from graphene by exposing graphene sheets to a “cold” hydrogen plasma. For example a low-pressure (0.1 mbar) hydrogen-argon mixture (10% H₂) with dc plasma ignited between two aluminum electrodes may be used (2 hours of plasma treatment to reach complete hydrogenation). An example, as described in [D. C. Elias et al, Science 323, 610 (2009), which is incorporated herein by reference].

Two-sided hydrogenation has been demonstrated on mechanically exfoliated suspended graphene membranes. Two-sided hydrogenation could be obtained using molecular spacers and proceeding to intercalation as in the case of graphite-intercalation compounds [see, for example, http://www.physorg.com/news188056335.html, which is incorporated herein by reference]. This also provides a way to manufacture graphane starting from bulk graphite, but it has not been demonstrated so far.

Graphene-based bulk materials can be produced by reduction of a colloidal suspension of exfoliated graphene oxide sheets in water with hydrazine hydrate [for example, S. Stankovich et al, Carbon 45, 1558 (2007), which is incorporated herein by reference]. (Volumetric adsorption of molecular hydrogen of this material has been demonstrated within the context of hydrogen storage research in [G. Srivanas et al, Carbon 48, 630 (2010), which is incorporated herein by reference], but no evidence of graphane was reported (the fabrication of graphane would require H plasma)).

Graphene-oxide frameworks have been announced in [www.physorg.com/print188056335.html, which is incorporated herein by reference]. Here the graphene sheets are separated by benzene-boronic acid spacers.

Fluorination may be performed as follows.

Fluorinated graphane, including carbon nanotubes, can be obtained by reaction of with fluorine gas, which is similar to the procedure developed for the fluorination of graphite [H. F. Bettinger, Chem. Phys. Chem. 4, 1283 (2003), which is incorporated herein by reference].

Fluorinated graphite is produced by high-temperature reaction of fluorine gas with graphite [see for instance D. E. Palin et al, Nature 162, 925 (1948), which is incorporated herein by reference].

Other examples are [O. Ruff, O. Bertschneider, F. Evert, Z. Anorg. Allg. Chem. 1, 217 (1934); Y. Kita, N. Watanabe, Y. Fujii, J. Am. Chem. Soc. 101, 3832 (1979), both of which are incorporated herein by reference]. Another procedure is for example disclosed in U.S. Pat. No. 4,859,444, also incorporated herein by reference.

Useful methods in the analysis of the products are as follows.

It is possible to establish the ratio of sp²-bonded C atoms to sp³-bonded C atoms in the products using X-ray Photoelectron spectroscopy [for example, R. Haerle et al, Phys. Rev. B 65, 045101 (2001), which is incorporated herein by reference].

The amount of boron doping achieved can be measured for instance by NMR [for example, see E. A. Ekimov et al, Nature 428, 542 (2004), which is incorporated herein by reference]. Other methods for estimating this is secondary ion mass spectroscopy, electrical transport, and Raman measurements [for example see E. Bourgeois et al., Phys. Rev. B 74, 094509 (2006), which is incorporated herein by reference].

### BRIEF DESCRIPTION OF THE FIGURES

Examples of the technology will now be described with reference to the accompanying Figures, of which:

FIGS. 1a, 1b and 1c show density of states and band diagrams for various materials;

FIGS. 2a and 2b show phonon dispersion and optical modes;

FIGS. 3a, 3b and 3c show phonon density of states diagrams;

FIGS. 4a and 4b show superconducting the electron-phonon coupling and critical temperature against doping level for various materials;

FIG. 5 shows the arrangement of the carbon atoms in graphene and related materials; and

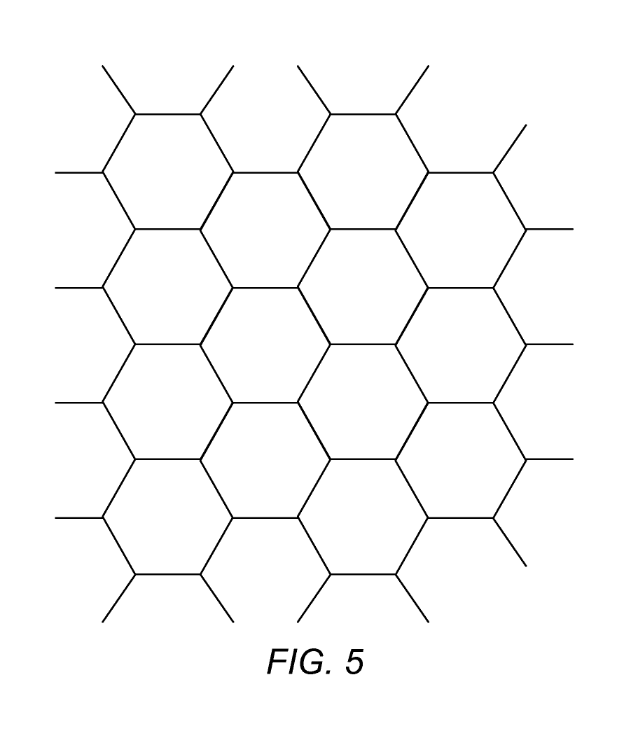

FIG. 6 shows a structure for applying an electric field to a material of the instant disclosure.

### DETAILED DESCRIPTION

Description herein is offered by reference to particular embodiments of the technology. It is to be understood that the nature and scope of the technology sought to be claimed is not limited to those specific embodiments but admits of variations that can be practiced by those of skill in the art.

### First and Second Embodiments

A first example is graphane. This is where all the carbon atoms in a sheet of hexagons are each bonded to a respective hydrogen atom. Hence the formula is (CH)ₙ where n is the number of carbon atoms in the sheet. (This formula may be inaccurate when taking into account the carbon atoms at the boundary of the sheet since carbons at the edge do not have three neighbouring carbons to bond to, which carbon atoms may have more than one bonded atom other than carbon.) Thus when considering this substance relative to graphene it may be said to be fully hydrogenated, fully saturated with hydrogen or fully substituted with hydrogen. Even if the material were to be prepared by routes other than from graphene this terminology would still apply to the final structure.

A second example is where only a proportion of the carbon atoms of the sheet has a hydrogen atom. Hence the formula is CₙHₓₙ, where n is the number of carbon atoms in the sheet and x is the proportion (up to but less than 1) of carbon atoms that have a hydrogen atom bonded to them. (This formula may be inaccurate when taking into account the carbon atoms at the boundary of the sheet since carbons at the edge do not have three neighbouring carbons to bond to, which carbon atoms may have more than one bonded atom other than carbon.) Thus when considering the structure of this substance relative to graphene it may be said to be partially hydrogenated, partially saturated with hydrogen or partially substituted with hydrogen (although of course no carbon is replaced). Even if the material were to be prepared by routes other than from graphene this terminology would still apply to the final structure.

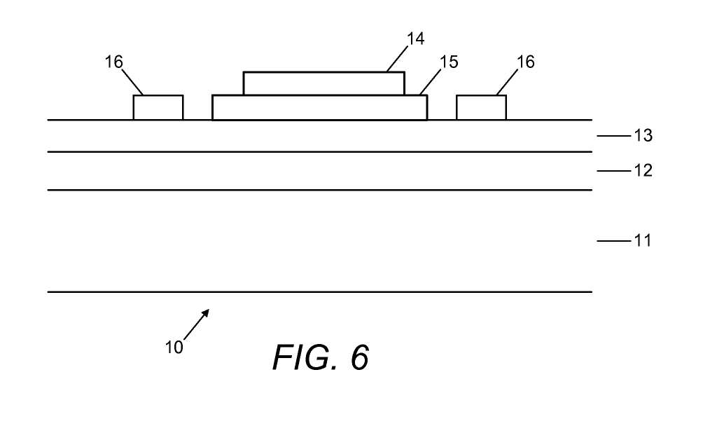

These materials may be rendered superconducting by the application of an electric field, for example by an electrode, which would commonly termed as a gate. The material is of course also cooled to below its superconducting critical temperature T_{c}. FIG. 6 shows an example of a suitable structure, which is analogous to a MOSFET transistor in semiconductor technology. A silicon substrate has a 11 has layer of silicon dioxide 12 thereon. The active layer 13 made of the hydrogenated graphene is on the oxide layer. The electrode 14, or gate, is a layer of metal thereon and separated and insulated from the active superconducting material by an insulating layer 15. Contacts 16 to pass a current along the active layer 13 of a metal such as gold or aluminium are provided.

Alternatively (or additionally) the materials are rendered superconducting by chemical doping, for example by substituting carbon atoms with boron.

### Third and Fourth Embodiments

A third example is fluorinated. This is where all the carbon atoms in a sheet of hexagons are each bonded to a respective fluorine atom. Hence the formula is (CF)ₙ where n is the number of carbon atoms in the sheet. (This formula may be inaccurate when taking into account the carbon atoms at the boundary of the sheet since carbons at the edge do not have three neighbouring carbons to bond to, which carbon atoms may have more than one bonded atom other than carbon.) Thus when considering this substance relative to graphene it may be said to be fully fluorinated, fully saturated with fluorine or fully substituted with fluorine. Even if the material were to be prepared by routes other than from graphene this terminology would still apply to the final structure.

A fourth example is where only a proportion of the carbon atoms of the sheet has a fluorine atom. Hence the formula is CₙFₓₙ, where n is the number of carbon atoms in the sheet and x is the proportion (up to but less than 1) of carbon atoms that have a fluorine atom bonded to them. (This formula may be inaccurate when taking into account the carbon atoms at the boundary of the sheet since carbons at the edge do not have three neighbouring carbons to bond to, which carbon atoms may have more than one bonded atom other than carbon.) Thus when considering this substance relative to graphene it may be said to be partially fluorinated, partially saturated with fluorine or partially substituted with fluorine (although of course no carbon is replaced). Even if the material were to be prepared by routes other than from graphene this terminology would still apply to the final structure.

These materials may be rendered superconducting by the application of an electric field, for example by an electrode, which would commonly termed as a gate. The material is of course also cooled to below its superconducting critical temperature T_{c}. FIG. 6 shows an example of a suitable structure, which is analogous to a MOSFET transistor in semiconductor technology. A silicon substrate has a 11 has layer of silicon dioxide 12 thereon. The active layer 13 made of the fluorinated graphene is on the oxide layer. The electrode 14, or gate, is a layer of metal thereon and separated and insulated from the active superconducting material by an insulating layer 15. Contacts 16 to pass a current along the active layer 13 of a metal such as gold or aluminium are provided.

Alternatively (or additionally) the materials are rendered superconducting by chemical doping, for example b substituting carbon atoms with boron.

### Fifth Embodiment

A diamond nano-rod doped with boron of 2 nm in diameter is prepared by CVD doped with boron.

### Sixth Embodiment

An undoped diamond nano-rod of 2 nm in diameter is provided with an electrode to which connected to a power supply to produce an electric field in the rod.

In all these embodiments the material is cooled with either liquid nitrogen or liquid helium to obtain superconductivity. Other forms of cryogenic apparatus could also be used.

### Further Examples

These materials can be used to form conductors. These may be used to convey power via electric currents, or to convey electric signals. These conductors can also be used to form superconducting magnets by, for example, being formed into coils and passing a current through them. Superconducting magnets find application in imaging systems, manipulation of ferrous objects and levitation devices (for example for trains and other vehicles).

The inventors have modelled fully hydrogenated and fully fluorinated graphene. The following discussion of their investigations is taken from their paper of 3 Feb. 2010 [arXiv:1002.0653v1 [cond-mat.mtrl-sci]] which deals with fully hydrogenated graphene or “graphane”. That paper, and also “First-Principles Prediction of Doped Graphane as a High-Temperature Electron-Phonon Superconductor” from Physical Review Letters [PRL 105, 037002 (2010)] of 14 Jul. 2010, are incorporated herein by reference. Numbered citations refer to the list of references appended to the description, before the claims.

Results obtained from similar investigations of fully fluorinated graphene are as follows immediately below. The markings *1 and *2 added in the text of the paper later mark the comparable results for graphane.

The electronic structure for fully fluorinated graphene is 2-dimensional as in graphane—the density of states at the top of the valence band is >0.4 states/eV/cell, which is about a factor 2 larger than in graphane. However the states at the top of the valence band in fully fluorinated graphene are mainly associated with fluorine atoms, therefore the electron-phonon coupling is smaller than in graphane and the superconducting pairing is correspondingly weaker.

The superconducting transition temperature is significantly smaller than in graphane, but can be just above the boiling point of liquid helium (4 K).

The discovery of superconductors such as magnesium diboride [1] and iron pnictides [2,3] opened new horizons in the landscape of superconductivity research, fueling renewed interest in the quest for high-temperature superconductivity in materials other than the copper oxides [4,5]. The critical temperature, T_{c}, reflects the energy scale of the quantum-mechanical interactions driving the electron condensation into the superconducting state [6]. In high-T_{c} copper oxides [7] the nature of the interaction leading to superconductivity is still under debate [8], yet it is generally accepted that Coulomb exchange and correlation effects, with energy scales around few hundred meVs, play an important role [9,10]. In contrast, in conventional superconductors the pairing is known to be driven by the interaction between electrons and lattice vibrations, with an associated energy scale of only a few ten meVs [11]. Due to the order-of-magnitude difference between such energy scales, it is generally assumed that conventional superconductors cannot exhibit T_{c} as high as copper oxides[4,5]. Here, we report first-principles calculations showing that p-doped graphane would make a conventional superconductor with T_{c} well above the boiling point of liquid nitrogen.

The Bardeen-Cooper-Schrieffer (BCS) theory [11] defines the basic theoretical framework to understand conventional superconductivity. Its generalization, known as the Migdal-Eliashberg theory [12], incorporating the lattice dynamics, provides a predictive computational tool. Within BCS, T_{c} is given by [11]:

k_{B}T_{c}=1.14 hω₀exp(−1/N_{F}V) (1)

where k_{B} is the Boltzmann constant, hω₀ a characteristic phonon energy, N_{F} the electronic density of states (EDOS) at the Fermi Energy, E_{F}, V an effective pairing potential resulting from the net balance between the attractive electron-phonon coupling (EPC) and the repulsive electron-electron interaction [11]. Even though the original BCS formula for T_{c} is now replaced by more refined expressions such as, e.g., the modified McMillan equation [13], Eq. (1) still proves useful for discussing trends. Eq. (1) indicates that one could maximize T_{c} by increasing the materials parameters ω₀, N_{F}, V. However, these are strongly intertwined, making such optimization complex [13,14]. Here, we propose a simple procedure, based on Eq. T_{c} to design a high-T_{c} superconductor.

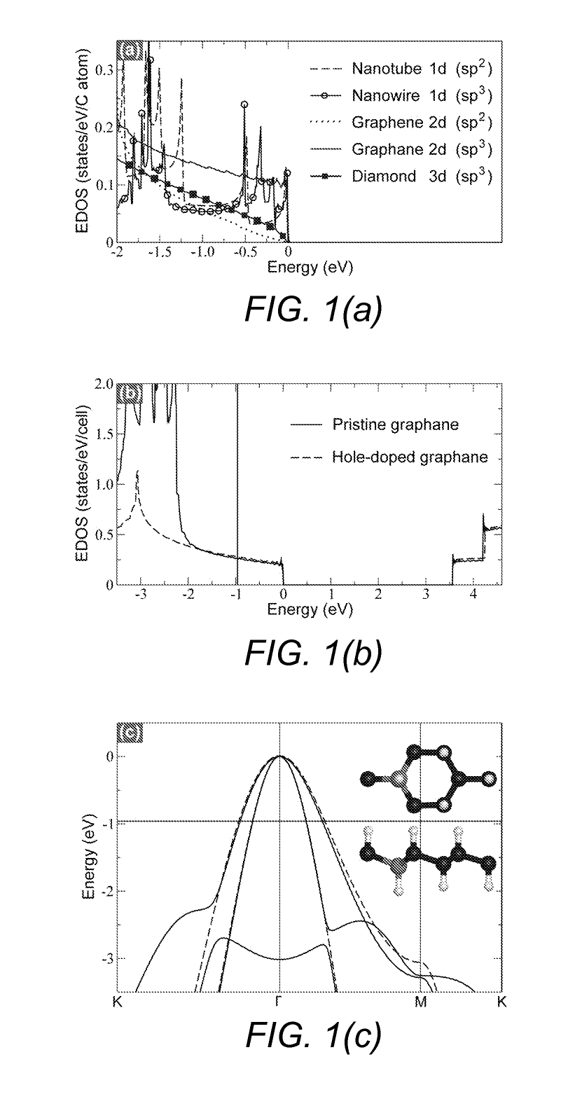

FIG. 1(a) is as follows. EDOS per carbon atom of 1d (nanotube; diamond nanowire), 2d (graphene; graphane) and 3d (diamond) systems. With the exception of graphene, with linear dispersions, the EDOS is proportional to E^{-1/2} in 1d, a step-like function in 2d, and E^{1/2} in 3d. The step-like EDOS in graphane implies that NF is large even at low doping. FIG. 1(b) is as follows. EDOS of pristine (solid black line) and 12.5% p-doped graphane (dashed red line). The top of the valence band is set as zero, and E_{F}=−0.96 eV (green line). The EDOS at E_{F} is similar in the two models (0.26 states/eV/cell in rigid-band and 0.27 states/eV/cell in supercell). FIG. 1(c) is as follows. Band structure of pristine (solid black line) and 12.5% p-doped graphane (dashed red line). (inset) Ball-and-stick 2×2 supercell with one substitutional B (top and side views).

Let us first consider the conventional superconductor with the highest T_{c}, MgB₂ (T_{c}=39 K)[1]. For simplicity, we neglect multi-band and anisotropy effects, which were the object of detailed investigations [15,16,17,18,19]. In MgB₂ the EPC contribution to V is large (˜1.4 eV, from λ=N_{F}V, using N_{F}=0.7 states/cell/eV, and λ ˜1 [16]) because the states with energy close to E_{F} (those which condensate in the superconducting state[11]) are of σ character, i.e., derive from bonding combinations of planar B sp² hybrids localised around the middle of B—B bonds [15,16,17,18,19]. These electronic states are significantly affected by the B—B bond length variation associated with bond-stretching E_{2g} phonons [20,16], resulting into a large EPC contribution to V. At the same time, the E_{2g} phonon energy is large (˜60 meV [16]), due to the small B mass, leading to a large ω₀ in Eq. (1). Furthermore, MgB₂ is a metal with a significant EDOS at E_{F}(˜0.7 states/cell/eV [16]). These three factors cooperate in Eq. (1) to establish a superconducting state with T_{c}=39 K [15,16,17,18,19]. However, many attempts to improve upon MgB₂, by investigating related materials, only met limited success [21], with the experimental T_{c} never higher than MgB₂.

We thus search for an alternative material having at least some of the desirable features of MgB₂ , i.e., (i) σ electrons at the Fermi surface,(ii) large bond-stretching phonon frequencies, and (iii) large EDOS at E_{F}. We note that the first two requirements are both met by B-doped diamond, a conventional BCS superconductor with T_{c}=4 K [22], where a small hole-like Fermi surface appears around the top of the valence band [23]. The electronic states at E_{F} have σ character deriving from the bonding combination of tetrahedral C sp3 hybrids, bearing some analogy to MgB₂. As these σ states are localized in the middle of the C—C bonds, they couple considerably to bond-stretching phonons [23,24], resulting in a large EPC contribution to V, even superior to MgB₂ (˜3 eV, from λ=N_{F}V, using N_{F}=0.1 states/cell/eV, and λ˜0.3 [25]) [26,27]. In addition, the light C atoms have high energy optical phonons (˜130 meV, even after softening induced by the large EPC [25,24]). However in B-doped diamond the EDOS at E_{F} is rather small (˜0.1 states/cell/eV for 2% doping [23, 28]). This compromises T_{c}. Thus, while B-doped diamond shares some of the desirable features of MgB₂, its 3-dimensional (3d) nature implies that the EDOS in proximity of the valence band scales as ˜E^{1/2} (with E measured from the valence band edge) [29], FIG. 1(a). Then, the number of carriers available for the superconducting state remains relatively small even for large doping. Superconducting diamond is thus a 3d analogue of MgB₂ [23,24].

This leads to the question of what would happen in a hypothetical B-doped diamond structure with reduced dimensionality, such as a thin film or a nanowire, where the EDOS can be significantly enhanced by quantum confinement. Indeed, the EDOS of a two-dimensional semiconductor goes as ˜θ(E) (θ being the step function) [29], hence the number of available carriers can be large, even at low doping. In order to estimate the expected EDOS increase in a diamond thin film it is helpful to consider a simple parabolic band model. For 2% B-doping, bulk diamond has N_{F}=0.1 states/eV/cell at E_{F}. A 0.5 nm thick diamond film with the same doping would have N_{F}˜0.5 states/eV/cell. Such an EDOS increase would significantly enhance T_{c}. Using the electron-phonon potential and the phonon frequency of bulk diamond, Eq. (1) gives that a 0.5 nm film would superconduct at T_{c}˜80 K. However the question remains whether it is possible to synthesize an atomically thin diamond film.

Recent work on graphene and its derivatives points to a positive answer. Soon after the discovery of graphene [30] several works considered how to functionalise and chemically modify this novel 2d material [31,35,36,32,33,34]. In particular, it was proposed that fully hydrogenated graphene (graphane) would be stable [37]. The main difference between graphene and graphane is that, while the former is fully sp² bonded, the latter is sp³, as diamond [37]. Recently, some experimental evidence of graphane was reported [38]. Since graphane is the 2d counterpart of diamond, our scaling arguments immediately point to doped graphane as a potential high-T_{c} superconductor. Doping could be achieved by gating, including using an electrolyte gate, or by charge-transfer, as done in graphene [31-34,39,40]. Substitutional doping of graphene was also reported, up to ˜10¹⁴ cm⁻² [35,41].

We thus perform density functional perturbation theory (DFPT) calculations of EPC and superconductivity in doped graphane within the framework of the Migdal-Eliashberg theory [12] and the local density approximation (LDA) [42,43]. By analogy with B doped diamond, we consider p-doping. This is simulated using the rigid-band approximation [44]. FIG. 1(b) shows that the calculated EDOS in p-doped graphane close to the valence band maximum follows a step-like behavior, as expected for a 2d system. At 3% doping the EDOS is 0.22 states/eV/cell, compared to 0.13 states/eV/cell in bulk diamond, with a factor 1.7 enhancement (*1—similar results for the fluorinated equivalent were given earlier hereinabove). FIG. 1(c) indicates that the dispersions close to E_{F} are essentially identical for a supercell containing B and for a rigid-band model of doped graphane. We expect this to hold also for lower doping, where the perturbation to the pristine dispersions is smaller. The similarity between these two models justifies our use of the rigid-band approximation. A supercell calculation with the B dopant explicitly included does not show impurity states inside the gap.

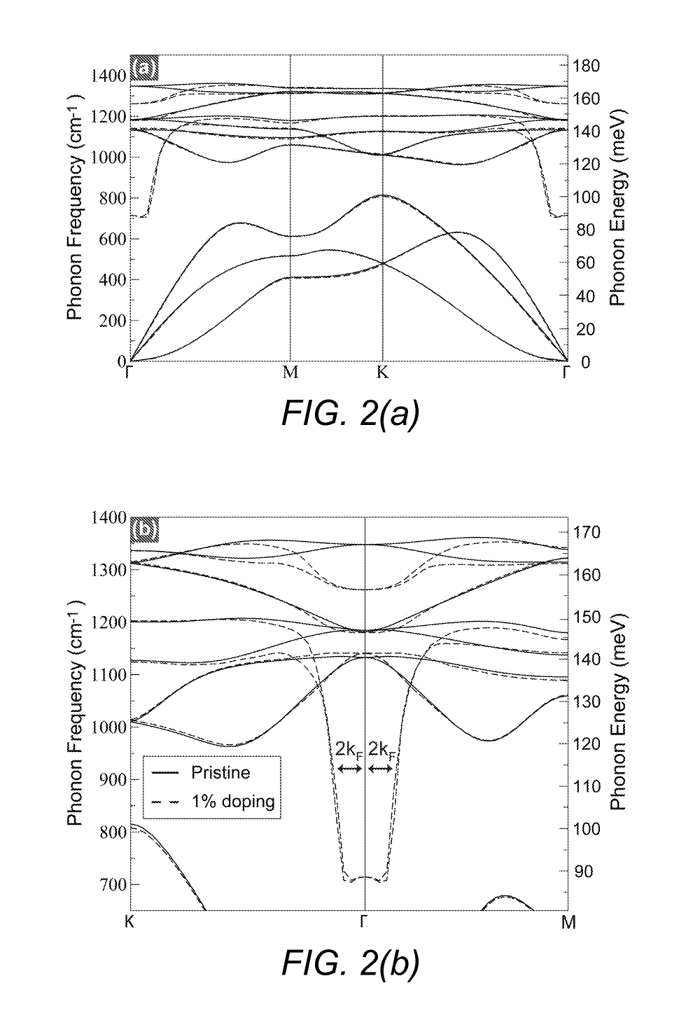

FIG. 2(a) is as follows. Phonon dispersion of pristine (solid black line) and 1% p-doped graphane (dashed blue lines). The C—H stretching modes have higher frequencies (2655-2711 cm⁻¹) and are not shown. FIG. 2(b) is as follows. Optical modes around the zone centre, showing the Kohn Anomalies. The horizontal (green) arrows indicate the average Fermi surface diameter.

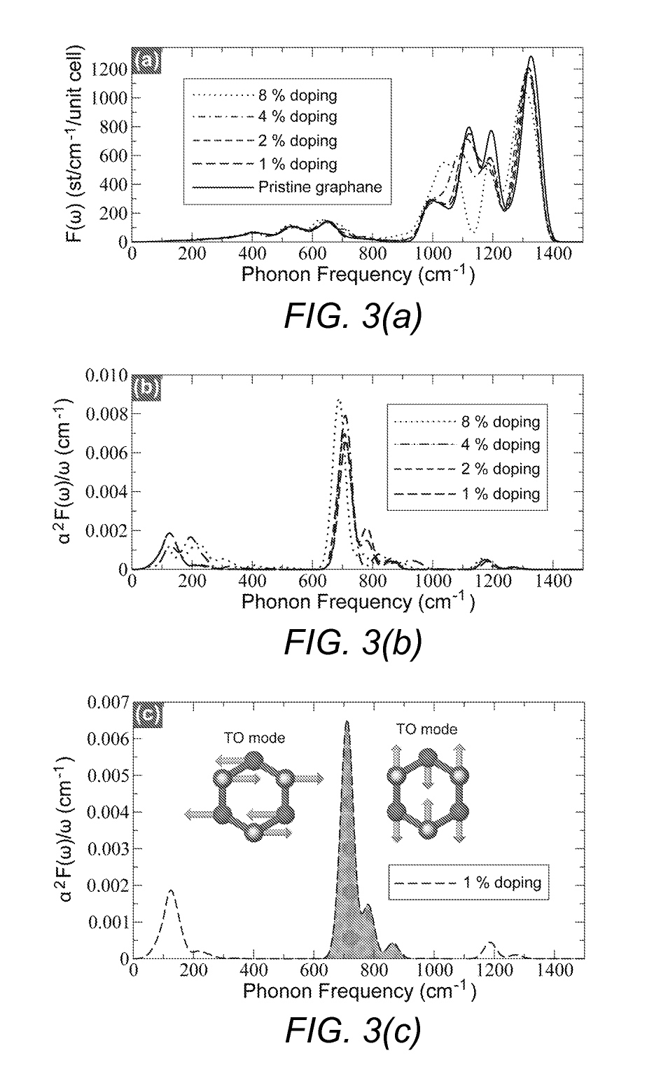

FIG. 3(a) is as follows. PDOS of pristine and doped graphane. FIG. 3(b) is as follows. Eliashberg function in p-doped graphane for increasing doping. The largest contribution comes from the optical modes, similar to diamond [24], but also the acoustic phonons couple to holes at the Fermi surface, similar to SiC [44]. FIG. 3(c) is as follows. Contributions to the Eliashberg function arising from the TO stretching modes (hashed region). (insets) Ball-and-stick representations of two TO modes. The arrows indicate the in-plane C—C stretching motions (carbons are shown in grey, hydrogens in white).

FIGS. 2(a) and 2(b) report the phonon dispersions of pristine and p-doped graphane and FIG. 3(a) the corresponding phonon density of states (PDOS). Upon doping, the optical zone-centre modes with in-plane C—C stretching soften as a result of the inception of Kohn Anomalies [45]. The two degenerate TO modes, having planar C—C stretching and H atoms moving in-phase with the C atoms, downshift from 1185 to 715 cm⁻¹ (147 to 89 meV). This is due to the large EPC of planar C—C stretching, which significantly affects the sp³-like electronic states at the Fermi surface. The two degenerate zone-centre modes, having in-plane C—C stretching and H atoms moving out-of-phase with respect to the C atoms, downshift from 1348 to 1257 cm⁻¹ (167 to 156 meV). The LO mode, with out-of-plane C—C stretching, does not couple to the electrons due to the different parity of potential and wavefunctions, resulting into a vanishing EPC. The two degenerate optical modes corresponding to the shear motion of the C and H planes (at ˜1133 cm⁻¹) and the C—H stretching modes (2 modes at 2663 and 2711 cm⁻¹) do not undergo softening upon doping. This is consistent with the electronic states associated with the C—H bonds having little weight at E_{F}, hence a small EPC.

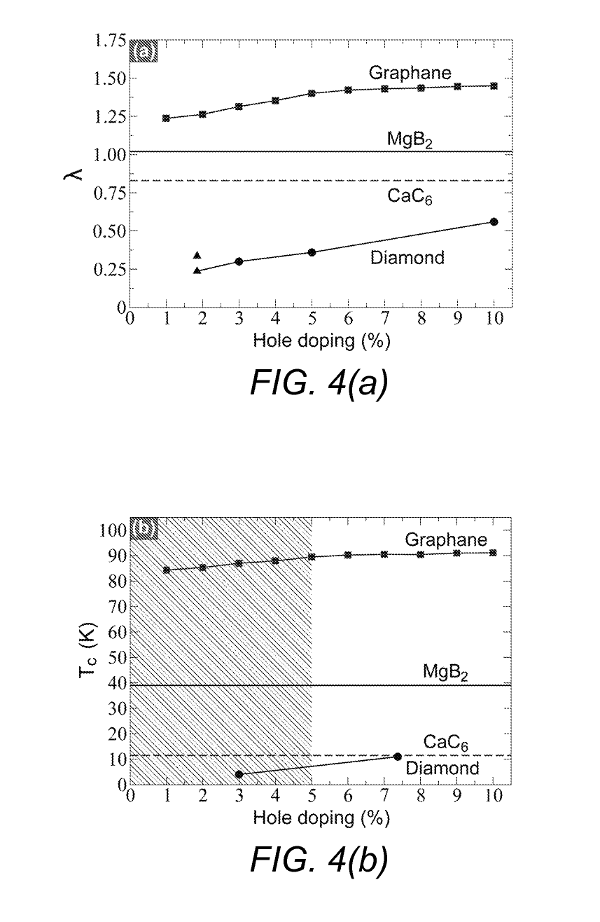

FIG. 4(a) is as follows. EPC of graphane as a function of doping, calculated using the standard DFPT formalism[51]: the Brillouin zone is sampled with an electron grid up to 300×300×1, smearing from 50 to 270 meV, and phonon grid of 100×100×1. For comparison, we plot literature values for MgB₂ (solid line [24]), CaC₆ (dashed line [52]), and diamond (solid line [24]; triangles[25]). More sophisticated calculations taking explicitly into account a substitutional dopant, such as B, could slightly change the EPCs [28,25]. However, in B-doped diamond a rigid-band model provides a lower EPC and a lower bound to T_{c} [25]. FIG. 4(b) is as follows. T_{c} calculated using the modified McMillan formula and a Coulomb pseudopotential μ*=0.13 [49]. The left-side hashed region indicates doping below the estimated MIT, where our formalism applies only to charge-transfer or gate-induced doping. Above MIT, it applies to substitutional doping as well. We use the isotropic Eliashberg formalism[49]. A more sophisticated description based on the fully anisotropic Eliashberg theory is expected to increase T_{c} [15,53]. For comparison we also show T_{c} of MgB₂ (solid line, T_{c}=39 K [1]), CaC₆ (dashed line, T_{c}=11.5 K [54]), and diamond (solid black line, T_{c}=4 K at ˜3% B [22]; 11 K at ˜7% B [55]

The softening of modes with a large C—C stretching component is similar to that reported in B-doped diamond [24,25]. In particular, the region of reciprocal space where the phonon softening is observed matches the diameter, 2k_{F}, of the hole Fermi surface around the Γ point, this being a typical signature of the Kohn effect[45]. The calculated phonon softening of the TO C—C stretching modes (˜58 meV or ˜470 cm⁻¹) is significantly larger than in other materials, as typical Kohn anomalies range from ˜5 meV (graphite and graphene[46]) to ˜10 meV (TaC [47]). In the case of B-doped diamond the phonon softening takes place through the creation of a non-dispersive defect branch associated with the B dopant [25]. A similar effect could happen in B-doped graphane, but we expect the magnitude of the doping-induced softening to be reasonably well described within our rigid-band model. Also, more sophisticated calculations, taking B explicitly into account[28,25] or with non-adiabatic corrections[48], may slightly revise the softening. Nevertheless, such a large softening stands out as a qualitative effect.

FIG. 3 plots the Eliashberg spectral function [49], which shows the relative contribution of different modes to the superconducting pairing [49].

α₂F(ω)=½Σ_{qv} ω_{qv}λ_{qv}δ(ω−ω_{qv}) (2)

We get that the TO in-plane C—C bond-stretching phonons with C and H atoms moving in-phase (see FIG. 3(c)) have the largest EPC, due to the σ character of the electronic states at EF and the large C displacements associated with these modes. This is similar to B-doped bulk diamond [23,24,25,28] and validates our hypothesis that p-doped graphane can be regarded as an atomically thin diamond film, exhibiting similar EPC and vibrational frequencies, but larger EDOS at EF. We note that the in-plane C—C bond-stretching phonons, with C and H atoms moving out-of-phase, do not contribute to the EPC. This happens because, upon softening, the four C—C planar stretching modes hybridize in such a way that those at 715 cm⁻¹ carry an increased weight on the C atoms, while the opposite happens for the two modes at 1257 cm⁻¹.

FIG. 4(a) plots the EPC as a function of doping, and FIG. 4(b) the corresponding T_{c}. We find that T_{c} exceeds the boiling point of liquid nitrogen, and falls within the same T_{c} range as copper oxides [50] (*2—similar results for the fluorinated equivalent were given earlier above). Due to the relatively constant EDOS below the top of the valence band [cf. FIG. 1(b)], T_{c} is rather insensitive to doping. This is important for the practical realization of superconducting graphane. Our results should be valid throughout the entire doping range considered here in the case of gate- or charge transfer-induced doping, since in these cases the holes are delocalized and doped graphane is in the metallic regime. On the other hand, for substitutional doping we expect our results to be valid only beyond the Mott metal-to-insulator transition (MIT). In absence of experimental MIT measurements in graphane, we estimate the critical doping concentration, n, using the following argument. In 3d the MIT occurs when the impurity wavefunctions are close enough that their overlap is significant [56]. For many materials a_{H}n^{c}^{1/3} ˜0.26, a_{H} being the radius of the ground-state wavefunction of an hydrogenic donor [56]. The radius can be calculated as a_{H}=εm*a₀/2, a₀ being the Bohr radius, ε the dielectric constant, and m* the effective mass [56]. In diamond a_{H}˜4 Å and n_{c}˜4*10²⁰ cm⁻³ [56], therefore the average separation between nearest neighbor B atoms is ˜15 Å. In the case of graphane we use a similar criterion, replacing the 3d hydrogenic impurity with a 2d one. The ground-state hydrogenic wavefuction in 2d has a radius a_{H}(2d)=εm*a₀/2 [57]. Using the dielectric constant and hole effective mass of diamond (ε=5.7$; m*=0.74) we find a_{H}(2d)=a_{H}/2 ˜2 Å. Thus, the average separation between nearest neighbor B atoms at the MIT is ˜7.5 Å, and the corresponding doping can be estimated as 5% B (1 B every 20 C atoms) or 2*10¹⁴ holes cm⁻². This could be feasible, considering that substitutional doping in graphene was reported up to 5% [41].

The calculated high-T_{c} for p-doped graphane bears consequences both for fundamental science and applications. One could envision hybrid superconducting-semiconducting circuits directly patterned through lithographic techniques, graphane-based Josephson junctions for nanoscale magnetic sensing, and ultimately an ideal workbench for exploring the physics of the superconducting state in two dimensions [58]. The superconducting phase transition in graphane could also be controlled by gating [34,59]. A high-T_{c} superconductor with gate-controllable T could lead to novel switching mechanisms in nanoscale field-effect transistors. Furthermore, the discovery of an electron-phonon superconductor with T_{c} above liquid nitrogen would mean that (i) there are no fundamental reasons to believe that BCS superconductors cannot have T_{c}>40 K (MgB₂), and (ii) high-T_{c} superconductivity does not take place exclusively in the copper oxides. In particular, our calculations indicate that at least one material could exist where a very strong EPC leads to T_{c} in the copper oxide range without triggering a lattice instability. The superconducting phase transition in systems with reduced dimensionality has been the subject of numerous theoretical studies [60,61]. Quantum fluctuations could destroy the superconducting order in 2d [62]. However, recent experimental evidence suggests that this is not necessarily the case [58,59,63]. In particular, for thin Pb it was reported that the superconducting state is robust down to two atomic layers [58]. Since our proposed mechanism of superconductivity in doped graphane is BCS-like, as in Pb, there should be no fundamental limits to prevent the realization of high-T_{c} superconductivity in graphane.

It is immediate to extend the present study to diamond nanowires, which have been the subject of intense investigations in the past few years [64]. For a 1d system the EDOS near a band edge has a van Hove singularity going as ˜E^{-1/2} [29]. We can assume phonon energies and EPC to be similar to bulk diamond and graphane. Then Eq. (1) would yield T_{c} as high as ˜150 K for a 1 nm nanowire (see EDOS in FIG. 1(a)). The possibility of achieving T_{c} higher than copper oxides by exploiting dimensionality deserves further investigation. Our work suggests that p-doped diamond nanostructures have an intriguing potential for high-T_{c} BCS-like superconductivity.

### REFERENCES

[1] J. Nagamatsu, N. Nakagawa, T. Muranaka, Y. Zenitani, J. Akimitsu, Nature 410, 63 (2001).

[2] Y. J. Kamihara, T. Watanabe, M. Hirano, H. Hosono, J. Am. Chem. Soc. 130, 3296 (2008).

[3] H. Takahashi, K. Igawa, K. Arii, Y. Kamihara, M. Hirano, H. Hosono, Nature 453, 376 (2008).

[4] J. E. Moussa, M. L. Cohen, Phys. Rev. B 74, 094520 (2006).

[5] P. A. Lee, N. Nagaosa, X. G. Wen, Rev. Mod. Phys. 78, 17 (2006).

[6] G. Tinkham, Introduction to superconductivity, McGraw-Hill, New York, (1996).

[7] J. G. Bednorz, K. A. Muller, Z. Phys. B 64, 189 (1986).

[8] F. Giustino, M. L. Cohen, S. G. Louie, Nature 452, 975 (2008).

[9] P. W. Anderson, Science 317, 1705 (2007).

[10] T. A. Maier, D. Poilblanc, D. J. Scalapino, Phys. Rev. Lett. 100, 237001 (2008).

[11] J. Bardeen, L. N. Cooper, J. R. Schrieffer, Phys. Rev. 108, 1175 (1957).

[12] P. B. Allen, B. Mitrovich, in Solid State Physics, Vol. 37 (eds. F. Seitz, F. Turnbull and H. Ehrenreich) 1-92, Academic, New York (1982).

[13] P. B. Allen, R. C. Dynes, Phys. Rev. B 12, 905 (1975).

[14] W. E. Pickett, J. Supercond. Novel Magn. 19, 291 (2006).

[15] H. J. Choi, D. Roundy, H. Sun, M. L. Cohen, S. G. Louie, Nature 418, 758 (2002).

[16] J. M. An, W. E. Pickett, Phys. Rev. Lett. 86, 4366 (2001).

[17] J. Kortus, I. I. Mazin, K. D. Belashchenko, V. P. Antropov, L. L. Boyer, Phys. Rev. Lett. 86, 4656 (2001).

[18] K. P. Bohnen, R. Heid, B. Renker, Phys. Rev. Lett. 86, 5771 (2001).

[19] A. Floris, G. Profeta, N. N. Lathiotakis, M. Luders, M. A. L. Marques, C. Franchini, E. K. U. Gross, A. Continenza, S. Massidda, Phys. Rev. Lett. 94, 037004 (2005).

[20] T. Yildirim, O. Gülseren, J. W. Lynn, C. M. Brown, T. J. Udovic, Q. Huang, N. Rogado, K. A. Regan, M. A. Hayward, J. S. Slusky, T. He, M. K. Haas, P. Khalifah, K. Inumaru, R. J. Cava, Phys. Rev. Lett. 87, 037001 (2001).

[21] H. J. Choi, S. G. Louie, M. L. Cohen, Phys. Rev. B 80, 064503 (2009).

[22] E. A. Ekimov, V. A. Sidorov, E. D. Bauer, N. N. Mel'nik, N. J. Curro, J. D. Thompson, S. M. Stishov, Nature 428, 542 (2004).

[23] K. W. Lee, W. E. Pickett, Phys. Rev. Lett. 93, 237003 (2004).

[24] L. Boeri, J. Kortus, O. K. Andersen, Phys. Rev. Lett. 93, 237002 (2004).

[25] F. Giustino, J. R. Yates, I. Souza, M. L. Cohen, S. G. Louie, Phys. Rev. Lett. 98, 047005 (2007).

[26] M. Calandra, F. Mauri, Phys. Rev. Lett. 101, 016401 (2008).

[27] X. Blase, E. Bustarret, C. Chapelier, T. Klein, C. Marcenat, Nature Mater. 8, 375 (2009).

[28] X. Blase, C. Adessi, D. Connetable, Phys. Rev. Lett. 93, 237004 (2004).

[29] A. D. Sutton, Electronic structure of materials, Oxford University Press, Oxford (1993).

[30] K. S. Novoselov, D. Jiang, F. Schedin, T. J. Booth, V. V. Khotkevich, S. V. Morozov, A. K. Geim, Proc. Natl. Acad. Sci. U.S.A. 102, 10451 (2005).

[31] A. Geim, K. Novoselov, Nature Mater. 6, 183 (2007).

[32] N. Jung, N. Kim, S. Jockusch, N. J. Turro, P. Kim, L. Brus, Nano Lett. 9, 4133 (2009).

[33] I. Gierz, C. Riedl, U. Starke, C. R. Ast, K. Kern, Nano Lett. 8, 4603 (2008).

[34] A. Das, S. Pisana, B. Chakraborty, S. Piscanec, S. K. Saha, U. V. Waghmare, K. S. Novoselov, H. R. Krishnamurthy, A. K. Geim, A. C. Ferrari, A. K. Sood, Nat. Nanotech. 3, 210 (2008).

[35] X. Wang, X. Li, L. Zhang, Y. Yoon, P. K. Weber, H. Wang, J. Guo, H. Dai, Science 324, 768 (2009).

[36] F. Cervantes-Sodi, G. Cs\'[a]nyi, S. Piscanec, A. C. Ferrari, Phys. Rev. B, 77, 165427 (2008).

[37] J. O. Sofo, A. S. Chaudari, G. Barber, Phys. Rev. B 75, 153401 (2007).

[38] D. C. Elias, R. R. Nair, T. M. G. Mohiuddin, S. V. Morozov, P. Blake, M. P. Halsall, A. C. Ferrari, D. W. Boukhvalov, M. I. Katsnelson, A. K. Geim, K. S. Novoselov, Science 323, 610 (2009).

[39] T. O. Wehling, K. S. Novoselov, S. V. Morozov, E. E. Vdovin, M. I. Katsnelson, A. K. Geim, A. I. Lichtenstein, Nano Lett. 8, 173 (2008).

[40] S. Y. Zhou, D. A. Siegel, A. V. Fedorov, A. Lanzara arXiv:cond-mat:0807.4791 (2008)

[41] X. Li, H. Wang, J. T. Robinson, H. Sanchez, G. Diankov, H. Dai, arXiv:0910.0862

[42] D. M. Ceperley, B. J. Alder, Phys. Rev. Lett. 45, 566 (1980).

[43] A. Zunger, J. P. Perdew, Phys. Rev. B 23, 5048 (1980).

[44] J. Noffsinger, F. Giustino, G. Louie, M. L. Cohen, Phys. Rev. B 79, 104511 (2009).

[45] W. Kohn, Phys. Rev. Lett. 2, 393 (1959).

[46] S. Piscanec, M. Lazzeri, F. Mauri, A. C. Ferrari, J. Robertson, Phys. Rev. Lett. 93, 185503 (2004).

[47] J. Noffsinger, F. Giustino, S. G. Louie, M. L. Cohen, Phys. Rev. B 77, 180507(R) (2008).

[48] A. M. Saitta, M. Lazzeri, M. Calandra, F. Mauri, Phys. Rev. Lett. 100, 226401 (2008).

[49] G. Grimvall, The electron-phonon interaction in metals, North-Holland, New York (1981).

[50] W. E. Pickett, Rev. Mod. Phys. 61, 433 (1989).

[51] S. Baroni, S. de Gironcoli, A. Dal Corso, P. Giannozzi, Rev. Mod. Phys. 73, 515 (2001).

[52] M. Calandra, F. Mauri, Phys. Rev. B 74, 094507 (2006).

[53] H. J. Choi, D. Roundy, H. Sun, M. L. Cohen, S. G. Louie, Phys. Rev. B 66, 020513(R) (2002).

[54] N. Emery, C. Hérold, M. d'Astuto, V. Garcia, Ch. Bellin, J. F. Marêché, P. Lagrange, G. Loupias, Phys. Rev. Lett. 95, 087003 (2005).

[55] K. Ishizaka, R. Eguchi, S. Tsuda, T. Yokoya, A. Chainani, T. Kiss, T. Shimojima, T. Togashi, S. Watanabe, C. T. Chen, C. Q. Zhang, Y. Takano, M. Nagao, I. Sakaguchi, T. Takenouchi, H. Kawarada, S. Shin, Phys. Rev. Lett. 98, 047003 (2007).

[56] E. Bustarret, P. Achatz, B. Sacépé, C. Chapelier, C. Marcenat, L. Ortéga, T. Klein, Phil. Trans. R. Soc. A 366, 267 (2008).

[57] X. L. Yang, S. H. Guo, F. T. Chan, K. W. Wong, W. Y. Ching, Phys. Rev. A 43, 1186 (1991).

[58] S. Qin, , J. Kim, Q. Niu, C. K. Shih, Science 324, 1314 (2009).

[59] J. T. Ye, S. Inoue, K. Kobayashi, Y. Kasahara, H. T. Yuan, H. Shimotani, Y. Iwasa, Nature Mater. doi:10.1038/nmat2587 (2009).

[60] T. M. Rice, Phys. Rev. 140, A1889 (1965).

[61] P. C. Hohenberg, Phys. Rev. 158, 383 (1966).

[62] W. J. Skocpol, M. Tinkham, Rep. Prog. Phys. 38, 1049 (1975).

[63] A. Gozar, G. Logvenov, L. F. Kourkoutis, A. T. Bollinger, L. A. Giannuzzi, D. A. Muller, I. Bozovic, Nature 455, 782 (2008).

[64] T. Yang, S. Berber, J. F. Liu. G. P. Miller, D. Tománek, J. Chem. Phys. 128, 124709 (2008).

[65] www.quantum-expresso.org

Each of the foregoing references is incorporated herein by reference in its entirety.

The foregoing description is intended to illustrate various aspects of the instant technology. It is not intended that the examples presented herein limit the scope of the appended claims. The invention now being fully described, it will be apparent to one of ordinary skill in the art that many changes and modifications can be made thereto without departing from the spirit or scope of the appended claims.

1. A doped, at least partially saturated graphene wherein the saturation is by hydrogen, or by halogen, or by hydrogen and halogen.

2. An at least partially saturated graphene as claimed in claim 1 which is fully saturated by saturation hydrogen and/or halogen.

3. An at least partially saturated graphene as claimed in claim 1 wherein the proportion of carbon atoms having a hydrogen and/or a halogen is above 75%.

4. An at least partially saturated graphene as claimed in claim 1 wherein the proportion of carbon atoms having a hydrogen and/or a halogen is above 90%.

5. An at least partially saturated graphene as claimed in claim 1 wherein the proportion of carbon atoms having a hydrogen and/or a halogen is above 95%.

6. An at least partially saturated graphene as claimed in claim 1 wherein the proportion of carbon atoms having a hydrogen and/or a halogen is above 99%.

7. An at least partially saturated graphene as claimed in claim 1 in the form of a sheet.

8. An at least partially saturated graphene as claimed in claim 1 in the form of a nano-tube.

9. An at least partially saturated graphene as claimed in claim 1 in the form of multiple layers of the at least partially saturated graphene.

10. An at least partially saturated graphene as claimed in claim 1 in the form of a ribbon.

11. An at least partially saturated graphene as claimed in claim 1 wherein the material is on a substrate.

12. An at least partially saturated graphene as claimed in claim 1, wherein the saturation is by hydrogen.

13. An at least partially saturated graphene as claimed in claim 1 wherein the saturation is by fluorine.

14. An at least partially saturated graphene as claimed in claim 1 wherein the hydrogen or halogen is on one side of the carbon sheet of the graphene.

15. An at least partially saturated graphene as claimed in claim 1 wherein the hydrogen or halogen is on different sides of the carbon sheet of the graphene.

16. An at least partially saturated graphene as claimed in claim 1 wherein the doping is chemical.

17. An at least partially saturated graphene as claimed in claim 16 wherein the doping is where carbon atoms are substituted by boron atoms.

18. An at least partially saturated graphene as claimed in claim 1 wherein the doping makes the material p-type.

19. An at least partially saturated graphene as claimed in claim 1 wherein the doping makes the material n-type.

20. An at least partially saturated graphene as claimed in claim 1 wherein the doping is by an electric field.

21. An at least partially saturated graphene at a temperature below its superconducting critical temperature.

22. A doped diamond nano-rod.

23. A doped diamond nano-rod at a temperature below its superconducting critical temperature.

24. A device comprising:
a layer of at least partially saturated graphene wherein the saturation is by hydrogen and/or halogen, and
a gate layer thereon.

25. A device as claimed in claim 24 comprising an insulating layer between the gate and the graphene layer.

26. A device as claimed in claim 24 at a temperature below the superconducting critical temperature of the at least partially saturated graphene.

27. A device comprising
a diamond nano-rod, and
a gate layer thereon.

28. A device as claimed in claim 27 comprising an insulating between the gate and the nano-rod.

29. A device as claimed in claim 27 at a temperature below the superconducting critical temperature of diamond nano-rod.

30. A substance having a carbon structure in which carbon atoms are bonded together by sp³ bonds, and which the carbon structure is low dimensional, exhibiting superconductivity.

31. A substance as claimed in claim 30 wherein the carbon atoms are arranged in sheets.

32. A substance as claimed in claim 31 wherein the sheets are nanotubes.

33. A substance as claimed in claim 30 wherein the carbon atoms are arranged in the diamond structure.

34. A substance as claimed in claim 30 at a temperature below its superconducting critical temperature.
